(19) United States
(12) Patent Application Publication
(10) Pub. No.: US 2024/0374825 A1
(43) Pub. Date: Nov. 14, 2024

(54) Damper Overfill Chamber to Increase Functional Robustness Regarding Variance in Parts and Processes

(71) Applicant: AMGEN INC., Thousand Oaks, CA (US)

(72) Inventors: Christian PLAMBECH, Soeborg (DK); Matias MELANDER, Copenhagen (DK); Knud Hellek FINKEN, Veksø Sjælland (DK); Martin Nissen ENEMARK, Syddanmark (DK)

(73) Assignee: AMGEN INC., Thousand Oaks, CA (US)

(21) Appl. No.: 18/660,595

(22) Filed: May 10, 2024

Related U.S. Application Data
(60) Provisional application No. 63/501,949, filed on May 12, 2023.

(51) Int. Cl. A61M 5/20

(52) U.S. Cl. CPC A61M 5/2033; A61M 2005/206; A61M 2005/208; A61M 2005/2086; A61M 2205/0222

(57) ABSTRACT

A damper mechanism for a drug delivery device comprising a frame member, a damper member operably coupled to a drive assembly of the drug delivery device, a damper fluid chamber formed between at least a portion of the frame member and the damper member, and a damper fluid disposed within the damper fluid chamber. The frame member and the damper member rotate relative to each other, and the damper fluid exerts an opposing force on at least one of the frame member and the damper member. The damper fluid chamber includes a main section and an overfill section. The overfill section receives excess damper fluid from the main section to reduce an effect a variation in fill level in the damper fluid chamber has on the opposing force exerted on at least one of the frame member or the damper member.

### CROSS-REFERENCE TO RELATED APPLICATIONS

Priority is claimed to U.S. Provisional Patent Application No. 63/501,949, filed May 12, 2023, the entire contents of which are hereby incorporated by reference herein.

### FIELD OF DISCLOSURE

The present disclosure generally relates to injectors and, more particularly, to a torque driven injector having a damping mechanism.

### BACKGROUND

Autoinjectors and on-body injectors offer several benefits in delivery of medicaments and/or therapeutics. One of the benefits can include simplicity of use, as compared with traditional methods of delivery using, for example, conventional syringes.

Many injector systems use coil spring structures to provide actuation energy for functions such as needle insertion and medicament delivery. The use of springs can offer benefits of simplicity for the user and device automation but can have certain limitations. For example, there is a linear relationship between force and displacement in linear spring actuators. To provide sufficient energy for drug delivery at the end of plunger stroke, an excessive amount of energy may be input to the system as drug delivery commences.

Further, as higher viscosity drugs are delivered via autoinjectors, requisite spring forces will likely increase. Springs with higher spring constants transmit more force per travel distance to the drug product and primary container at the beginning of travel. In many autoinjectors, an air gap is present between a plunger face and a storage portion that contains the medicament prior to its injection into a user. When the drug is to be administered, the spring urges the plunger face through the air gap towards the medicament. Because the plunger face exhibits little resistance when traversing the air gap and due to large forces urging the plunger, the plunger face may make abrupt contact with the storage portion containing the medicament. A patient may feel this excessive energy as a “slap” or similar physical “bump”, as the spring driven plunger impacts the stopper of the primary container storing the drug. Further, the user may also experience a jerk, recoil, and/or a reaction force when rotational movement begins due to the abrupt change in acceleration. Such mechanical bumps can be distracting and/or disturbing to users of the injectors and can, therefore, impact proper dose administration. Further, the “slap” and “bump” generated by the excessive energy can potentially cause catastrophic effects, such as breakage of the primary container and drug product damage caused by shear load. Furthermore, high force springs can produce undesirably high shear rates on the drug product.

Further still, patients may experience a large variation in injection times due to variations in characteristics of the medicament. These variations can be disturbing to users, who may think something is wrong with administration of the drug, and thus they may end the injection before they receive the full dosage. Variations in injection time may be caused by large drug viscosity variation due to changes to the temperature of the drug, large variations in friction between components in the device (e.g., between a syringe barrel and a stopper), and so on.

### SUMMARY

In accordance with a first aspect of the present disclosure, a damper mechanism for a drug delivery device includes a frame member, a damper member operably coupled to a drive assembly of the drug delivery device, a damper fluid chamber formed between at least a portion of the frame member and the damper member, and a damper fluid disposed within the damper fluid chamber. Upon activating the drive assembly of the drug delivery device, the frame member and the damper member rotate relative to each other, and the damper fluid exerts an opposing force on at least one of the frame member and the damper member. The damper fluid chamber includes a main section and an overfill section. The overfill section is configured to receive excess damper fluid from the main section to reduce an effect a variation in fill level in the damper fluid chamber has on the opposing force exerted on at least one of the frame member and the damper member. The main section has a first thickness defined between an inner surface of the frame member and an outer surface of the damper member, and the overfill section has a second thickness defined between the inner surface of the frame member and the outer surface of the damper member that is greater than the first thickness of the main section.

In some embodiments, the frame member may extend circumferentially around the longitudinal axis to define a cavity, and the frame member may comprise an end wall and a cylindrical protrusion that extends axially away from the end wall relative to a longitudinal axis to a terminal end spaced apart axially from the end wall. In other embodiments, the cylindrical protrusion of the frame member may comprise a base section that extends axially away from the end wall and a tip section that extends axially away from the base section to the terminal end. The base section of the cylindrical protrusion of the frame member may have a first thickness, and the tip section of the cylindrical protrusion of the frame member may have a second thickness that is less than the first thickness of the base section. In some embodiments, the damper member may comprise a body that extends circumferentially around the longitudinal axis to define the outer surface of the damper member, and a thickness of the body proximate the main section may be greater than a thickness of the body proximate the overfill section. In some embodiments, the damper member may be located in the cavity formed by the frame member to form the damper fluid chamber between at least the portion of the frame member and the damper member.

In some embodiments, the damper member may comprise a body that extends circumferentially around the longitudinal axis to define the outer surface of the damper member, an outer ring that extends circumferentially around the body and spaced apart radially from the body relative to the longitudinal axis to define a channel therebetween, and a band member that extends radially between and interconnects the body and the outer ring to form a bottom of the channel. The cylindrical protrusion of the frame member may extend axially from the housing into the channel between the outer surface of the body and an inner surface of the outer ring, and the damper fluid chamber may be defined between the outer surface of the body and the inner surface of the frame member. In some embodiments, the terminal end of the frame member may be spaced apart axially from the band member of the damper member to define a gap therebetween. In some embodiments, the gap may be free of any excess damper fluid. In other embodiments, a space defined directly between the frame member and the outer ring of the damper member may be free of excess damper fluid.

In some embodiments, the body of the damper member may comprise an end wall spaced apart axially from the frame member to define a portion of the damper fluid chamber, and an annular wall that extends axially away from the end wall and extends circumferentially around the longitudinal axis to define a bore. In some embodiments, the bore may be configured to receive a portion of the drive assembly of the drug delivery device.

In other embodiments, the damper member may comprise a body that extends circumferentially around the longitudinal axis to define the outer surface of the damper member, an outer ring that extends circumferentially around the body and spaced apart radially from the body relative to the longitudinal axis to define a channel therebetween, and a band member that extends radially between and interconnects the body and the outer ring to form a bottom of the channel. In this aspect, the cylindrical protrusion of the frame member may extend axially from the housing into the channel between the outer surface of the body and an inner surface of the outer ring, and the damper fluid chamber may be defined between the outer surface of the body and the inner surface of the frame member. In some embodiments, the body of the damper member may comprise an end wall spaced apart axially from the frame member to define a portion of the damper fluid chamber, and an annular wall that extends axially away from the end wall to the band member and extends circumferentially around the longitudinal axis to define a bore. In some embodiments, the body of the damper member may further comprise an extension that extends axially from the end wall of the body opposite the annular wall and engages the frame member.

In some embodiments, the housing of the drug delivery device may comprise a housing shell that extends along the longitudinal axis, and a housing end cap coupled to the housing shell opposite the proximal end. The frame member may be formed integrally with the housing end cap of the housing. In other embodiments, the overfill section of the damper fluid chamber may be coated with a low vicious intermediate fluid to serve as a lubricating boundary layer reducing the resulting torque of the excess damper fluid.

In accordance with another aspect of the present disclosure, a drug delivery device comprises a housing having a proximal end, a distal end, and a longitudinal axis extending between the proximal end and the distal end thereof and a needle assembly at least partially disposed within the housing at the proximal end thereof. The needle assembly comprises a syringe barrel containing a medicament and a needle or a cannula. The drug delivery device also comprises a drive assembly at least partially disposed within the housing and operably coupled to the needle assembly to urge the medicament through the needle or cannula and a damper mechanism at least partially disposed within the housing adjacent to the distal end thereof. The damper mechanism is operably coupled to the drive assembly and the housing, and upon activating the drive assembly, the damper mechanism dampens an effect thereof. The damper mechanism comprises a frame member, a damper member operably coupled to the drive assembly, a damper fluid chamber formed between at least a portion of the frame member and the damper member, and a damper fluid disposed within the damper fluid chamber. Upon activating the drive assembly of the drug delivery device, the frame member and the damper member rotate relative to each other, and the damper fluid exerts an opposing force on at least one of the frame member and the damper member. Additionally, the damper fluid chamber includes a main section and an overfill section. The overfill section is configured to receive excess damper fluid from the main section to reduce an effect a variation in fill level in the damper fluid chamber has on the opposing force exerted on at least one of the frame member and the damper member. The main section has a first thickness defined between an inner surface of the frame member and an outer surface of the damper member, and the overfill section has a second thickness defined between the inner surface of the frame member and the outer surface of the damper member that is greater than the first thickness of the main section.

In some embodiments, the frame member may comprise an end wall that defines a portion of the distal end of the housing, and a cylindrical protrusion that extends axially away from the end wall toward the proximal end relative to the longitudinal axis to a terminal end spaced apart axially from the end wall. The cylindrical protrusion of the frame member may vary in thickness as the cylindrical protrusion extends axially away from the end wall to the terminal end. In other embodiments, the cylindrical protrusion of the frame member may comprise a base section that extends axially away from the end wall, and a tip section that extends axially away from the base section to the terminal end. The base section of the cylindrical protrusion of the frame member may have a first thickness, and the tip section of the cylindrical protrusion of the frame member may have a second thickness that is less than the first thickness of the base section.

In yet another embodiment, the damper member may comprise a body that extends circumferentially around the longitudinal axis to define the outer surface of the damper member, an outer ring that extends circumferentially around the body and spaced apart radially from the body relative to the longitudinal axis to define a channel therebetween, and a band member that extends radially between and interconnects the body and the outer ring to form a bottom of the channel. The frame member may extend axially from the housing into the channel between the outer surface of the body and an inner surface of the outer ring, and the damper fluid chamber may be defined between the outer surface of the body and the inner surface of the frame member. In some embodiments, the terminal end of the frame member may be spaced apart axially from the band member of the damper member to define a gap therebetween. In some embodiments, the gap may be free of any excess damper fluid, and wherein there is no excess damper fluid in a space defined between the frame member and the outer ring of the damper member. In other embodiments, the housing of the drug delivery device may comprise a housing shell that extends along the longitudinal axis and a housing end cap coupled to the housing shell opposite the proximal end, and the frame member may be formed integrally with the housing end cap of the housing.

Additional features of the present disclosure will become apparent to those skilled in the art upon consideration of the embodiments exemplifying the best mode of carrying out the disclosure as presently perceived.

### BRIEF DESCRIPTIONS OF THE DRAWINGS

The above needs are at least partially met through provision of the drug delivery device comprising a damper mechanism described in the following detailed description, particularly when studied in conjunction with the drawings, wherein:

FIG. 1 illustrates a cross-sectional view of an example torque driven drug delivery device having a damper mechanism in accordance with various embodiments of the present disclosure;

FIG. 2 illustrates a detailed cross-sectional view of the damper mechanism of the drug delivery device of FIG. 1 in accordance with various embodiments of the present disclosure;

FIG. 3 illustrates another detailed cross-sectional view of the damper mechanism of FIGS. 1 and 2 in accordance with various embodiments of the present disclosure;

FIG. 4 illustrates a detailed view of a portion of the damper mechanism of FIGS. 1-3 in accordance with various embodiments of the present disclosure;

FIG. 5 illustrates a detail view of another portion of the damper mechanism of FIGS. 1-4 in accordance with various embodiments of the present disclosure;

FIG. 6A illustrates an exploded view of the damper mechanism of FIGS. 1-5 before the damper member is operably coupled to the frame member in accordance with various embodiments of the present disclosure;

FIG. 6B illustrates a view of the damper mechanism of FIG. 6A after the damper member is operably coupled to the frame member in accordance with various embodiments of the present disclosure;

FIG. 7 illustrates a graph showing the performance of the damper mechanism of FIGS. 1-5, 6A, and 6B in relation to the fill level of the damper fluid in accordance with various embodiments of the present disclosure;

FIG. 8A illustrates a cross-sectional view of the damper mechanism of FIGS. 1-5, 6A, and 6B when the damper fluid included in the damper mechanism has a nominal fill level in accordance with various embodiments of the present disclosure;

FIG. 8B illustrates a cross-sectional view of the damper mechanism of FIGS. 1-5, 6A, and 6B when the damper fluid included in the damper mechanism has a low fill level in accordance with various embodiments of the present disclosure;

FIG. 8C illustrates a cross-sectional view of the damper mechanism of the drug delivery device of FIG. 1 showing the damper fluid included in the damper mechanism has a high fill level in accordance with various embodiments of the present disclosure;

FIG. 9A illustrates a cross-sectional view of a prior embodiment of a damper mechanism for a drug delivery device when a damper fluid included in the damper mechanism has a nominal fill level;

FIG. 9B illustrates a cross-sectional view of the prior embodiment of the damper mechanism of FIG. 9A when the damper fluid included in the damper mechanism has a low fill level;

FIG. 9C illustrates a cross-sectional view of the prior embodiment of the damper mechanism of FIG. 9A when the damper fluid included in the damper mechanism has the high fill level;

FIG. 10A illustrates a cross-sectional view of a prior embodiment of a damper mechanism for a drug delivery device when a damper fluid included in the damper mechanism has a nominal fill level;

FIG. 10B illustrates a cross-sectional view of the prior embodiment of the damper mechanism of FIG. 10A when the damper fluid included in the damper mechanism has a low fill level;

FIG. 10C illustrates a cross-sectional view of the prior embodiment of the damper mechanism of FIG. 10A when the damper fluid included in the damper mechanism has a high fill level;

FIG. 11 illustrates variations in fill level caused by varying a relative position between a frame member and a damper member of a drug delivery device in accordance with various embodiments of the present disclosure;

FIG. 12 illustrates a graph depicting shear stress as a function of shear rate in accordance with various embodiments of the present disclosure;

FIG. 13 illustrates a graph depicting apparent viscosity as a function of shear rate in accordance with various embodiments of the present disclosure;

FIG. 14 illustrates a perspective view of an example drug delivery device having a clearance between components in accordance with various embodiments of the present disclosure;

FIG. 15 illustrates an illustration of an example of the effect of a damper mechanism on drug expulsion in accordance with various embodiments of the present disclosure;

FIG. 16 illustrates an illustration of an example of the effect of a damper mechanism on drug expulsion in a low-friction environment in accordance with various embodiments of the present disclosure;

FIG. 17 illustrates an illustration of an example of the effect of a damper mechanism on drug expulsion in a high-friction environment in accordance with various embodiments of the present disclosure; and

FIG. 18 illustrates example model calculations for a drug delivery device in accordance with various embodiments of the present disclosure.

### DETAILED DESCRIPTION

Generally speaking, pursuant to these various embodiments, a torque driven injector includes a housing, a syringe assembly containing a medicament to be injected into a user, and a rotatable actuating assembly using a torque spring to cause the medicament to be injected into the user. As the rotatable actuating assembly rotates to cause the drug to be administered, a fluid damper is used to provide a more consistent drug delivery time between drugs of varying viscosities, as well as drugs that may exhibit changes in viscosity based on different environmental changes (e.g., varying temperatures).

Further, as the actuating mechanism rotates, the damper mechanism can reduce or eliminate the “slap” or “bump” that occurs when the plunger face first contacts the medicament and/or medicament storage device. The damper mechanism may also reduce the “jerk” or recoil when the mechanism is released. Accordingly, a user will not feel this sudden movement during the drug delivery process and can comfortably and safely administer the medicament. Further, the torque spring, which uses a high number of turns, discussed in further detail below, may maintain near-constant start and end torque as compared to traditional springs and those with fewer turns. As a result, smaller autoinjectors may be used, which can increase overall user comfort. Additionally, the damper may reduce and/or eliminate the variation in injection times and minimize the risk of the device stalling. The damper may also provide for design freedom to target optimal injection times for usability and can potentially eliminate the need to customize the device for different drug volumes.

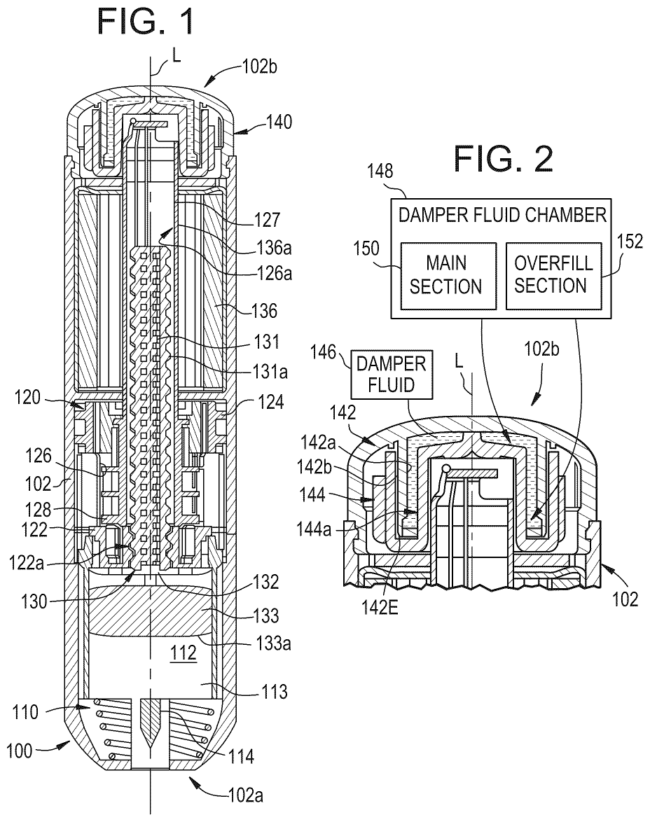

Referring now to the drawings, an exemplary autoinjector 100 is illustrated in FIG. 1. As shown in FIG. 1, the autoinjector 100 includes a housing 102, a needle assembly 110 at least partially disposed within the housing 102, a drive assembly 120 also at least partially disposed within the housing 102, and a damper mechanism 140 also at least partially disposed within the housing 102 as shown in FIG. 1. The housing 102 defines a proximal end 102a, a distal end 102b, and defines a longitudinal axis “L” extending between the proximal end 102a and the distal end 102b.

The needle assembly 110 is generally disposed at or near the proximal end 102a of the housing 102 includes a syringe barrel 112 containing a medicament 113 and a needle or a cannula 114 as shown in FIG. 1. The needle assembly 110 may include any number of additional components such as, for example, a sidewall or sidewalls, openings to allow the medicament 113 to pass to the needle or cannula 114, return springs, shield members, filter members, and the like, but for the sake of brevity, will not be discussed in substantial detail. A portion of the syringe barrel 112 may be open to accommodate a portion of the drive assembly 120, which will be described in further detail below. The syringe barrel 112 may be of any desired shape and/or size to accommodate various quantities of medicament 113. In some examples, the syringe barrel 112 can be constructed from a polymeric material such as cyclic-olefin polymer (“COP”), cyclic olefin copolymer (“COC”), or a glass material. Other examples are possible.

The drive assembly 120 is at least partially disposed within the housing 102 and is operably coupled to the needle assembly 110 as shown in FIG. 1. The drive assembly 120 is operably coupled to the needle assembly 110 to urge the medicament through the needle or cannula 114. The drive assembly 120 includes a drive mechanism 136 in the form of a torque or power spring 136. The drive mechanism 136 exerts a force on the other components of the drive assembly 120 to cause the medicament to move through the needle assembly 110.

In some embodiments, the damper mechanism 140 may be at least partially disposed within the housing 102 at the distal end 102b thereof, for example, as shown in FIGS. 1 and 2. As shown in FIGS. 1 and 2, the damper mechanism 140 is operably coupled to a portion of the drive assembly 120 (e.g., the plunger rod guide 126) and the housing 102. The damper mechanism 140 acts to dampen the effect of the torque spring 136 on the drive assembly 120 by exerting an opposite force on the drive assembly 120.

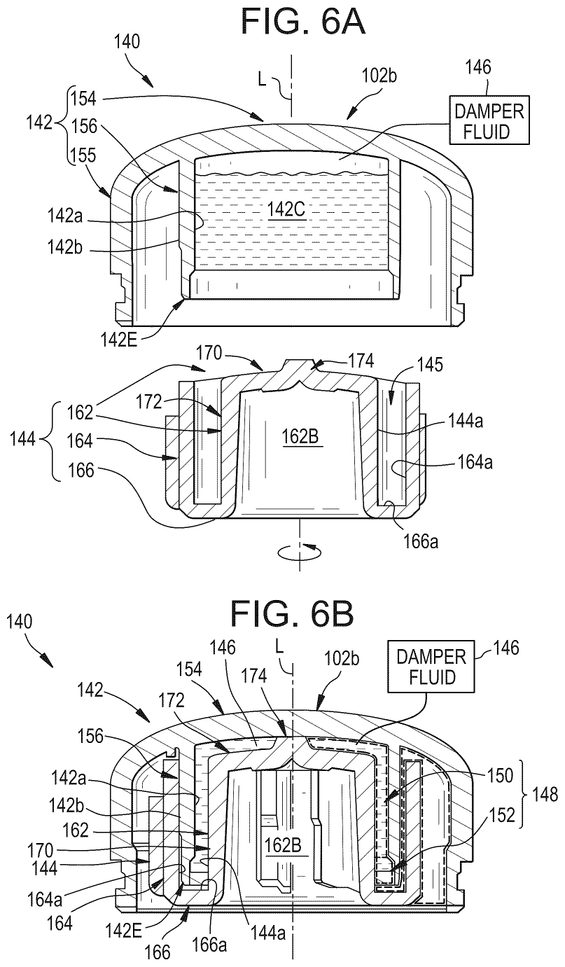

The damper mechanism 140 includes a frame member 142, a damper member 144, and a damper fluid 146 disposed in a damper fluid chamber 148 formed between the frame member 142 and the damper member 144 as shown in FIGS. 2-5. As used herein, a damper fluid may refer to a damping or dampening grease, gel, oil, fluid, or any combination thereof. For example, a damper fluid may comprise a damping or dampening gel made of silicone, such as NyeMed® 7328 manufactured by NYE Lubricants that has high viscosity. The frame member 142 extends circumferentially around the longitudinal axis L to define a cavity 142C (shown in FIG. 6A). The damper member 144 is operably coupled to the drive assembly 120 included in the drug delivery device 100. In some embodiments, for example, the damper member 144 may be located in the cavity 142C (shown in FIG. 6A) formed by the frame member 142 to form the damper fluid chamber 148. Upon activating the drive assembly 120 of the drug delivery device 100, the frame member 142 and the damper member 144 rotate relative to each other and the damper fluid 146 exerts an opposing force on at least one of the frame member 142 and the damper member 144.

Relative rotation between components of the damper mechanism 140 causes the damper fluid 146 to dampen the force applied by the drive mechanism 136 of the drive assembly 120. Specifically, in this example, the damper member 144 rotates relative to the frame member 142 when the drive assembly 120 rotates. A torque from the torque spring 136 exists between the damper member 144 and the frame member 142, thereby causing the system to accelerate from rest thus increasing speed. During relative rotation, the damper fluid 146 in the damper fluid chamber 148 between the damper member 144 and the frame member 142 experiences shear stress due to rotation of the damper member 144. The damper fluid 146 thus exerts an opposite acting reaction torque on the drive assembly 120 and, in particular, a plunger rod guide 126 of the drive assembly 120. The speed of the drive assembly 120 increases until the opposite acting damper torque has been built up to the same level as the dosing torque and equilibrium is reached. This equilibrium occurs at a specific speed and torque and is dependent on a number of factors such as, for example, geometry of the damper mechanism 140, fluid properties of the damper fluid 146, and the torque profile of the torque spring 136.

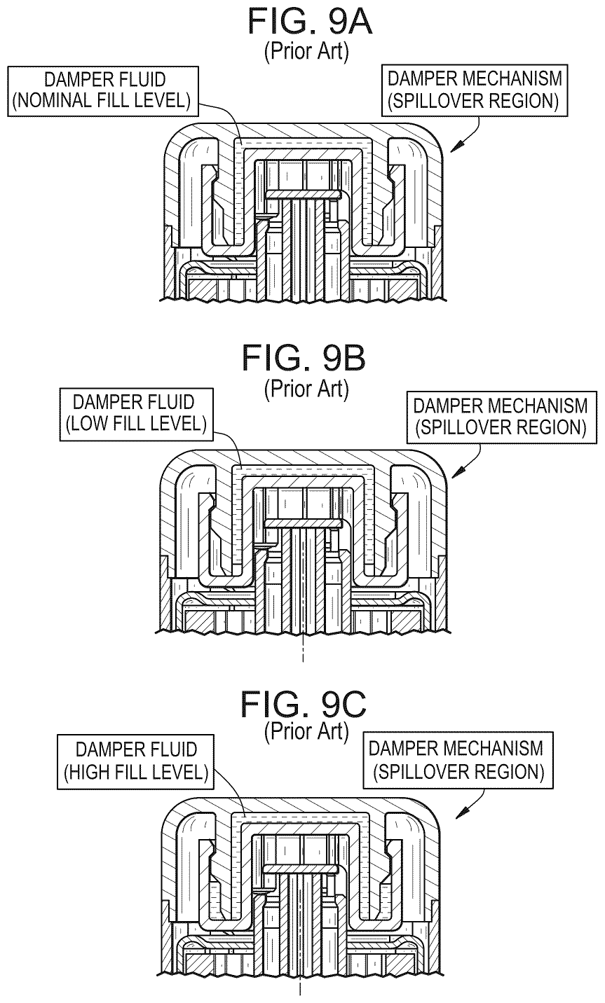

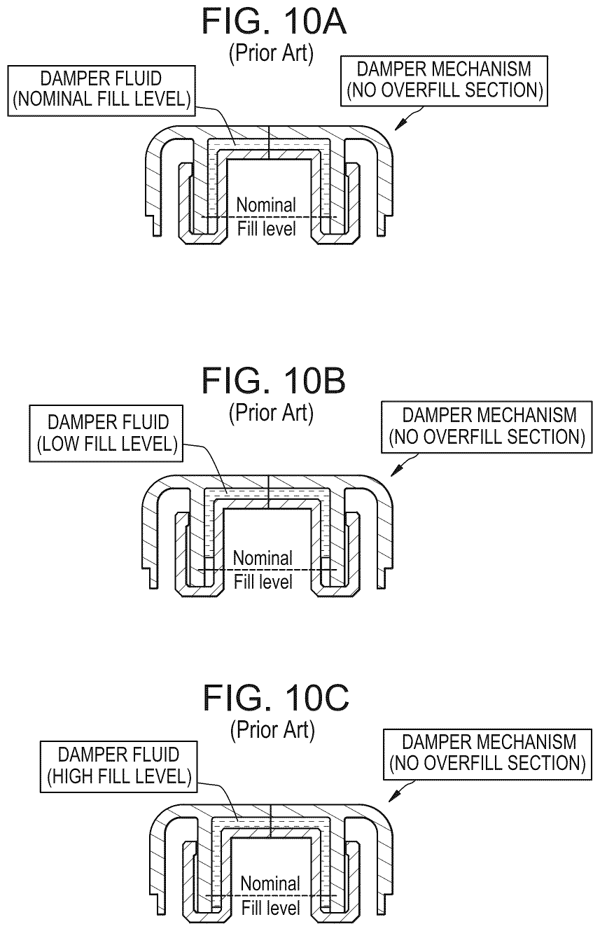

However, in prior embodiments of the damper mechanism, such as the damper mechanism shown in FIGS. 9A-C and 10A-C, variation of fill level in the damper fluid chamber may result in an inadequate amount or an excess amount of damper fluid in the channel. This variation in fill level may affect the opposing force applied to the drive assembly. For example, not enough damper fluid (as shown in FIGS. 9B and 10B) may result in not enough friction and not enough opposing force applied to the drive assembly, and too much damper fluid (as shown in FIGS. 9C and 10C) may result in too much friction and too much opposing force applied to the drive assembly. Additionally, excess damper fluid may cause the damper fluid 146 to escape the damper fluid chamber 148 and move to other parts inside and/or outside the housing 102.

Referring back to FIGS. 2-5, to limit the uneven distribution of the damper fluid 146, the damper fluid chamber 148 includes a main section 150 and an overfill section 152 in fluid communication with the main section 150. The overfill section 152 is configured to receive excess damper fluid from the main section 150 to reduce an effect that a variation in fill level in the damper fluid chamber 148 and/or the excess damper fluid have on the opposing force exerted on at least one of the frame member 142 and the damper member 144. The main section 150 has a first thickness 150T defined between an inner surface 142a of the frame member 142 and an outer surface 144a of the damper member 144, while the overfill section 152 has a second thickness 152T defined between the inner surface 142a of the frame member 142 and the outer surface 144a of the damper member 144. The second thickness 152T of the overfill section 152 is greater than the first thickness 150T of the main section 150, for example, as shown in FIGS. 2-4.

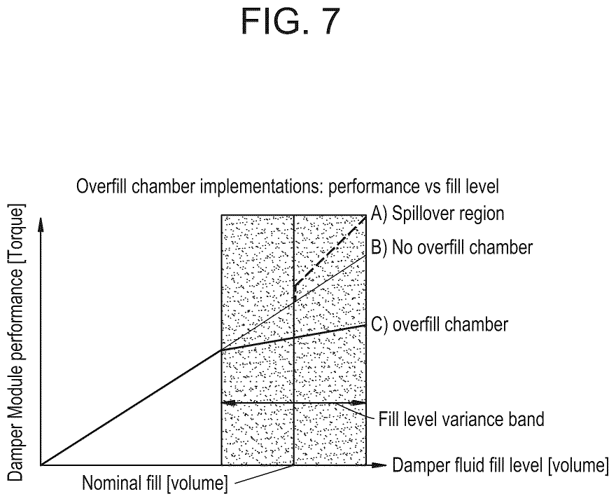

The overfill section 152 of the damper fluid chamber 148 is sized so that excess damper fluid 146 (i.e., variation in fill level in the damper fluid chamber 148) does not increase/minimally increases the resulting damper torque. The increased thickness 152T of the overfill section 152 compared to the main section 150 reduces the shear rate, as shown in FIG. 7, by reducing the contact area of the excess damper fluid 146 with the inner surface 142a and/or the outer surface 144a. Thus, the resulting specific torque contribution of the excess damper fluid is reduced.

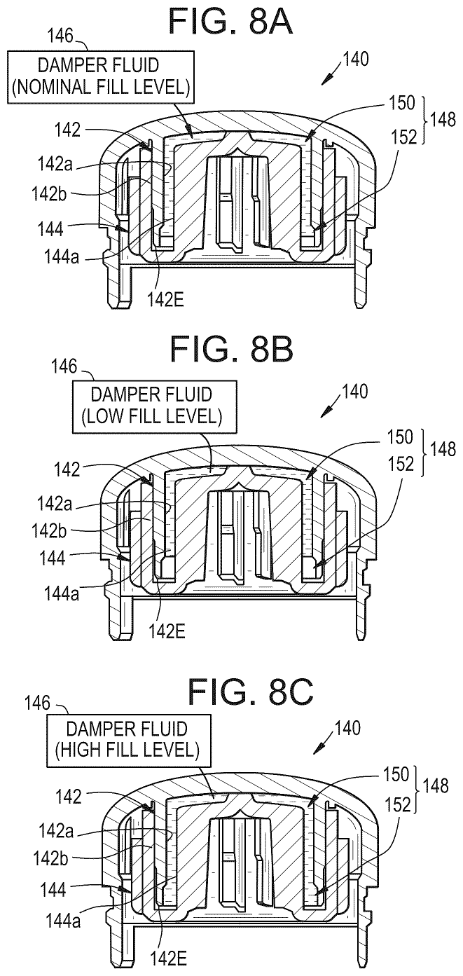

In FIGS. 9A-9C, a prior embodiment of a damper mechanism for a drug delivery device is shown. The damper mechanism of FIGS. 9A-9C has a spillover region, and excess damper fluid may flow into the spillover region. Accordingly, as shown in FIG. 7, the damper mechanism with a spillover region of FIGS. 9A-9C causes the torque applied by the damper mechanism to significantly increase, for example, compared to the damper mechanism 140 of FIGS. 8A-8C that does not have a spillover region. As discussed above, variation in fill level can significantly affect the opposing force or torque applied by the damper mechanism to the drive assembly. For example, as shown in FIG. 9B, having a low fill level or not enough damper fluid may result in not enough friction and not enough opposing force applied to the drive assembly. Additionally, as shown in FIG. 9C, having a high fill level or too much damper fluid may result in too much friction and significantly increase the torque applied to the drive assembly. Accordingly, a damper mechanism with a spillover region, as shown in FIGS. 9A-9C, can significantly affect the torque applied to the drive assembly compared to a damper mechanism without a spillover region, as shown in FIGS. 8A-8C.

Unlike the prior embodiment shown in FIGS. 9A-9C, where the damper mechanism has a spillover region, the damper mechanism 140, in accordance with the embodiments of the present disclosure, provides the overfill section 152 to reduce the amount of excess damper fluid flowing out of the damper fluid chamber 148. In some embodiments, the overfill section 152 may prevent excess damper fluid from flowing out of the damper fluid chamber 148. This may prevent the damper torque from increasing significantly, for example as shown in FIG. 7, because the overfill section is not farther away from the center axis L. Unlike the spillover region shown in FIGS. 9A-9C, the overfill section 152 of the damper fluid chamber 148 does not increase the moment arm length and/or the shear rate in the fluid compared to fluid in the main section 150 of damper fluid chamber 148.

Additionally, FIGS. 10A-10C illustrate a prior embodiment of a damper mechanism with no overfill section. Similar to the damper mechanism shown in FIGS. 9A-9C, variation in fill level in a damper mechanism with no overfill section, as shown in FIGS. 10A-10C, can also significantly affect the opposing force or torque applied to the drive assembly. For example, as shown in FIG. 10B, having a low fill level or not enough damper fluid may result in not enough friction and not enough opposing force applied to the drive assembly. Additionally, as shown in FIG. 10C, having a high fill level or too much damper fluid may result in too much friction and significantly increase the torque applied to the drive assembly. Accordingly, a damper mechanism with no overfill section, as shown in FIGS. 10A-10C, can significantly affect the torque applied to the drive assembly compared to a damper mechanism 140 with an overfill section 152, as shown in FIGS. 8A-8C.

Unlike the prior embodiment of a damper mechanism with no overfill section shown in FIGS. 10A-10C, the damper mechanism 140, in accordance with the embodiments of the present disclosure, provides the overfill section 152 to minimize the increase in the damper torque, for example as shown in FIG. 7. Compared to the prior embodiment of the damper mechanism with no overfill section as shown in FIGS. 10A-10C, the excess damper fluid 146 minimally affects the resulting damper torque regardless of the variation in fill level in the damper fluid chamber 148. The variation in fill level in the damper fluid chamber 148, for example as shown in FIGS. 8A-8C, may only minimally affect or not affect the resulting damper torque compared to the other prior embodiments of the damper mechanism shown in FIGS. 9A-9C and 10A-10C.

As shown in FIGS. 2-5, the overfill section 152 of the damper fluid chamber 148 ends at a terminal end 142E of the frame member 142. The overfill section 152 is sized so that excess damper fluid in the damper fluid chamber 148 does not extend past the terminal end 142E of the frame member 142. By sizing the overfill section 152 so that no excess damper fluid 146 flows out of the damper fluid chamber 148, i.e., the no fluid zone 151, excess damper fluid 146 is prevented from flowing onto/between other components within the device 100 so that the friction between the other components is not increased.

In some embodiments, the overfill section 152 of the damper fluid chamber 148 may be coated with a low vicious intermediate fluid. The overfill section 152 of the damper fluid chamber 148 may be coated with a low vicious intermediate fluid to serve as a lubricating boundary layer reducing the resulting torque of the excess damper fluid 146.

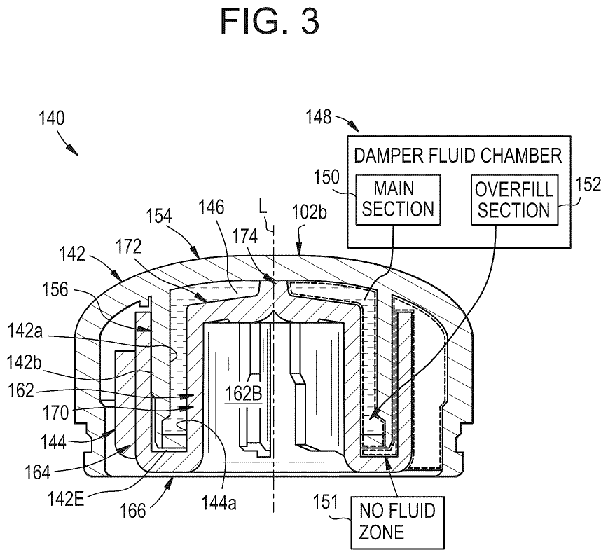

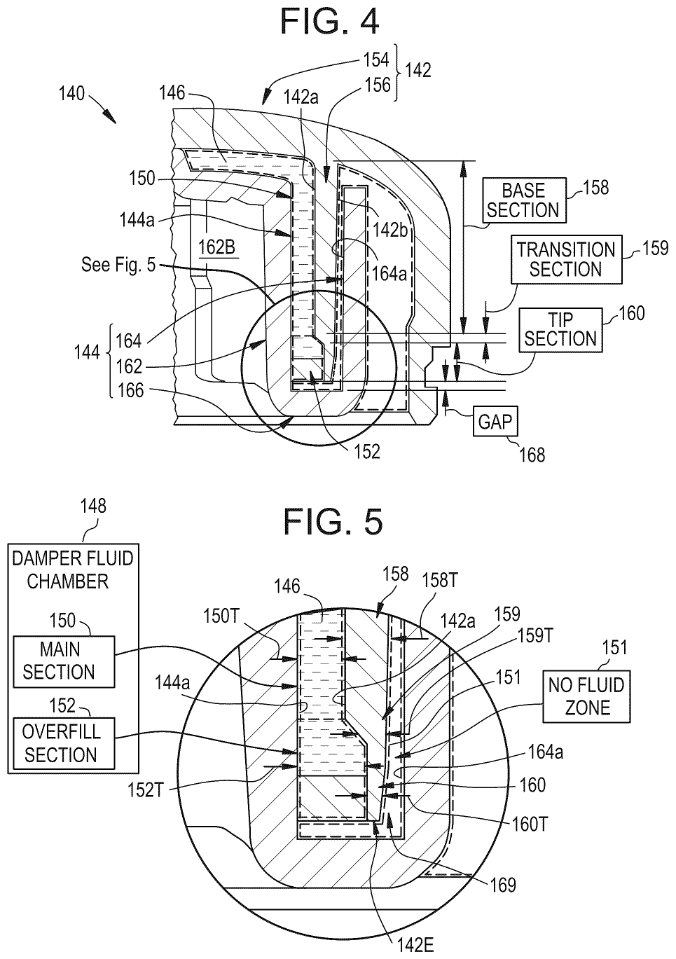

The frame member 142 includes an end wall 154 and a cylindrical protrusion 156 as shown in FIGS. 3 and 4. In some embodiments, the fame member 142 may be integrally formed with the housing 102 and the end wall 154 of the frame member 142 forms a portion of the distal end 102b of the housing 102. In other embodiments, the frame member 142 may be operably coupled to the housing 102 via any number of approaches such as, for example, adhesives, threaded, frictional connections, or the like. The cylindrical protrusion 156 extends axially away from the end wall 154 relative to the longitudinal axis L to the terminal end 142E spaced apart axially from the end wall 154.

As shown in FIGS. 2-5, 6A, and 6B, the frame member 142 may be formed integrally with the housing end cap of the housing 102. The housing 102 includes a housing shell 102 that extends along the longitudinal axis L and the housing end cap, i.e., the frame member 142. The housing end cap is coupled to the housing shell 102 opposite the proximal end 102a of the housing 102. In some embodiments, the frame member 142 further includes an outer shell 155 that extends circumferentially around the longitudinal axis L radially outward of the cylindrical protrusion 156. The outer shell 155 may couple to the housing shell 102 opposite the proximal end 102a.

In some embodiments, the end wall 154 and the cylindrical protrusions 156 define the inner surface 142a of the frame member 142, while the cylindrical protrusion 156 defines an outer surface 142b of the frame member 142, for example, as shown in FIGS. 3 and 4. The inner surface 142a faces the damper member 144, while the outer surface 142b is opposite the inner surface 142a and faces away from the damper fluid chamber 148.

In some embodiments, the cylindrical protrusion 156 of the frame member 142 varies in thickness as the cylindrical protrusion 156 extends axially away from the end wall 154 to the terminal end 142E, for example, as shown in FIGS. 2-5. The damper member 144 may have a constant thickness, as shown in FIGS. 2-5. In other embodiments, the cylindrical protrusion 156 may have a constant thickness as the cylindrical protrusion 156 extends axially away from the end wall 154 to the terminal end 142E, while the damper member 144 may have the variable thickness. For example, instead of the thickness of the cylindrical protrusion 156 decreasing in thickness as the cylindrical protrusion 156 extends axially away from the end wall 154 to the terminal end 142E, the thickness of the body 162 of the damper member 144 may decrease in thickness along the end wall 170 as the end wall 170 extends axially down the longitudinal axis L from the main section 150 to the overfill section 152. Accordingly, the thickness of the body 162 of the damper member 144 near the main section 150 may be greater than the thickness of the body 162 near the overfill section 152, while the thickness of the cylindrical protrusion 156 remains constant. In other embodiments, both the cylindrical protrusion 156 and the damper member 144 may have variable thicknesses as they extend axially along the longitudinal axis L.

As shown in FIG. 4, the cylindrical protrusion 156 of the frame member 142 may include a base section 158 and a tip section 160. The base section 158 extends axially away from the end wall 154 and the tip section 160 extends axially away from the base section 158 to the terminal end 142E. The base section 158 of the cylindrical protrusion 156 of the frame member 142 has a first thickness 158T and the tip section 160 of the cylindrical protrusion 156 of the frame member 142 has a second thickness 160T that is less than the first thickness 158T of the base section 158 as shown in FIG. 5. The first thickness 158T of the base section 158 and the second thickness 160T of the tip section 160 are both defined between the inner surface 142a and the outer surface 142b of the frame member 142.

In some embodiments, the cylindrical protrusion 156 of the frame member 142 further comprises a transition section 159, for example, as shown in FIG. 4. The transition section 159 extends between and interconnects the base section 158 and the tip section 160 of the cylindrical protrusion 156 of the frame member 142. As shown in FIG. 5, the transition section 159 may have a varying thickness 159T. The third thickness 159T of the transition section 159 is also defined between the inner surface 142a and the outer surface 142b of the frame member 142.

In some embodiments, the base section 158 of the cylindrical protrusion 156 of the frame member 142 has a first axial length 158, and the tip section 160 of the cylindrical protrusion 156 of the frame member 142 has a second axial length 160, for example, as shown in FIG. 4. The second axial length 160 of the tip section 160 is less than the first axial length 158 of the base section 158.

The transition section 159 of the cylindrical protrusion 156 of the frame member 142 has a third axial length 159 as shown in FIG. 4. The third axial length 159 is less than the first and second axial lengths 158, 160 of the base and tip sections 158, 160.

The damper member 144 includes a body 162, an outer ring 164, and a band member 166, as shown in FIGS. 3 and 4. The body 162 extends circumferentially around the longitudinal axis L to define a bore 162B that receives a portion of the drive assembly 120. In some embodiments, the body 162 defines the outer surface 144a of the damper member 144. The outer ring 164 extends circumferentially around the body 162 and is spaced apart radially from the body 162 relative to the longitudinal axis L to define a channel 145 therebetween. The band member 166 extends radially between and interconnects the body 162 and the outer ring 164 to form a bottom of the channel 145.

In some embodiments, the cylindrical protrusion 156 of the frame member 142 may be inserted into the channel 145 between the outer surface 144a of the body 162 and an inner surface 164a of the outer ring 164 and, thus, may extend axially into the channel 145, for example, as shown in FIG. 6A. The damper fluid chamber 148 is defined between the outer surface 144a of the body 162 and the inner surface 142a of the frame member 142. The band member 166 defines a bottom surface 166a that forms a part of the channel 145 along with the inner surface 164a of the outer ring 164 and the outer surface 144a of the body 162.

In some embodiments, the terminal end 142E of the frame member 142 may be spaced apart axially from the band member 166 of the damper member 144 to define a gap 168 therebetween, for example, as shown in FIG. 4. The gap 168 may be free of any excess damper fluid. By way of example, the gap 168 may be a no fluid zone 151.

In some embodiments, a space 169 defined directly between the frame member 142 and the outer ring 164 of the damper member 144 may be free of excess damper fluid, for example, as shown in FIG. 5. For example, the space between the outer surface 142b of the frame member 142 and an inner surface 164a of the outer ring 164 may be a no fluid zone 151. The body 162 of the damper member 144 includes an end wall 170, an annular wall 172, and an extension 174, as shown in FIGS. 3, 6A, and 6B. The end wall 170 is spaced apart axially from the frame member 142 to define a portion of the damper fluid chamber 148. The annular wall 172 extend axially away from the end wall 170 and extends circumferentially around the longitudinal axis L to define the bore 162B that receives the drive assembly 120 of the drug delivery device 100. The extension 174 extends axially from the end wall 170 of the body 162 opposite the annular wall 172 and engages the frame member 142. The extension 174 spaces the annular wall 172 apart from the frame member 142 to define the portion of the damper fluid chamber 148.

In some embodiments, the outer surface 144a of the body 162 of the damper member 142 extends along the end wall 170 and the annular wall 172, for example, as shown in FIG. 3. The damper fluid chamber 148 may extend radially and axially.

FIGS. 6A and 6B illustrate an exemplary method of assembling the damper mechanism 140 in accordance with various embodiments of the present disclosure. To assemble the damper mechanism 140, damper fluid 146 may be located in the cavity 142C of the frame member 142 before the damper member 144 is inserted into the cavity 142C. The damper member 144 may then be inserted into the cavity 142C of the frame member 142. In some embodiments, the body 162 of the damper member 144 may be inserted into the cavity 142C to cause the cylindrical protrusion 156 to extend into the channel 145 between the body 162 and the outer ring 164 of the damper member 144.

While the damper member 144 is then inserted into the cavity 142C of the frame member 142, the damper member 144 may be rotated about the longitudinal axis L, as shown in FIG. 6A. Rotation of the damper member 144 during insertion into the frame member 142 disperses the damper fluid 146 in the damper fluid chamber 148 formed between the frame member 142 and the damper member 144, for example, as shown in FIG. 6B.

Turning again to the drive assembly 120 of FIG. 1, the drive assembly 120 may include a nut 122 positioned adjacent to the syringe barrel 112, a trigger ring 124, a plunger rod guide 126, a plunger rod assembly 130, and the drive mechanism in the form of a torque or power spring 136. Generally, portions of the drive assembly 120 may be fixedly coupled to the housing 102 via any number of approaches. In some arrangements, the nut 122 may be formed integrally with the housing 102 and may include a threaded opening 122a. The trigger ring 124 selectively engages the nut 122 and is configured to move in an axial direction. In the illustrated example, the trigger ring 124 is in the form of a generally cylindrical ring having a generally circular inner surface and any number of ledges, protrusions, and grooves disposed around and/or inside the circumference of the ring. The trigger ring 124 may be coupled to the housing 102 via any number of techniques.

The plunger rod guide 126 includes a rod portion 127 and a base portion 128 coupled thereto. The plunger rod guide 126 includes an opening 126a extending at least partially through the rod portion 127 and the base portion 128. The base portion 128 can have any number of projections or tabs extending therefrom to define a slidable engagement with the trigger ring 124.

The plunger rod assembly 130 includes a plunger rod 131, a washer 132, and a plunger 133 that are moveable along the longitudinal axis L of the housing 102. The plunger rod 131 has a threaded portion 131a which is threadably coupled to the plunger rod guide 126 and the threaded opening 122a of the nut 122. The washer 132 minimizes frictional losses between rotation of the plunger rod 131 and the non-rotating plunger 133. In some approaches, the washer 132 may also be used to adjust the volume of medicament 113 by making the washer 132 thicker or narrower. Accordingly, the washer 132 may be used to accommodate a range of fill volumes of medicament 113 in the same device 100, thereby allowing for better control of the air gap between the bottom of the washer 132 and the top of the plunger 133.

The rod portion 127 of the plunger rod guide 126 is coupled to the plunger rod assembly 130 via any number of approaches including, for example, via a splined connection or slotted arrangement that allows for the plunger rod assembly 130 to be axially displaced relative to the plunger rod guide 126. As such, the plunger rod guide 126 guides rotational movement of the plunger rod assembly 130. The threaded portion 131a of the plunger rod 131, and correspondingly, the threaded opening 122a of the nut 122 may have a thread pitch suitable for any desired drug delivery rate or force/torque combination when driven by the drive mechanism 136. Relative rotation between the plunger rod 131 and the nut 122 causes the plunger rod 131 to advance axially towards the proximal end 102a of the housing 102. The plunger 133 has a top face 133a that is disposed near the syringe barrel 112.

In the illustrated example, the drive mechanism 136 is in the form of a power spring or a torque spring 136 having an inner portion 136a coupled to the rod portion 127 of the plunger rod guide 126 via any known approach to exert a torque on the plunger rod guide 126 that causes the plunger rod guide 126 to rotate about axis L. In some examples, the torque spring 136 may have a high number of turns to provide an appropriate rotational travel required to expel the medicament from the syringe barrel 112, however, additional parameters of the spring design may influence its torque output such as material properties and any applied heat treatments. The pre-shaping of the torque spring 136 may also impact its performance. As an example, in an autoinjector, a pre-stressed spring may be preferred, because the pre-stressing process generally increases torque output of the spring by initial coiling the spring in an opposite direction of the intended working condition, thereby causing permanent deformation in the steel band. This deformation maximizes the stresses in the material, thereby causing the torque to increase. Such an increase in torque is beneficial to minimize device size and weight.

In some examples, the torque spring 136 may have between approximately 1 and approximately 30 turns in the wound or loaded configuration, and preferably, approximately 12 turns. In some examples, the total spring turns may be higher due to a margin in both ends of the working range of approximately 20%, which may result in the range being between approximately 1*1.4=1.4 to 30*1.4=42. The dose mechanism turns are derived from the pitch and the required travel length. As previously stated, a smaller pitch is preferred due to requiring a low torque input and activation force. Accordingly, the activation force also will be lower. If a high axial force is not needed, the pitch can be raised and require fewer spring turns, thus allowing the device to be smaller. In some examples, the torque spring 136 may have a number of initial or preload turns to have a usable torque. After the preload turns, the torque spring 136 is further wound with working turns or turns that are used in the device during injection. As a non-limiting example, the torque spring 136 may have approximately 2.5 preload turns and approximately 6 working turns. As such, the total number of turns during assembly is approximately 8.5. However, due to potentially large tolerances in the angular positioning of spring terminations, the torque spring 136 may have an initial play before reaching a solid state, and thus may have a total of approximately 10 turns. Devices having different drug volumes and viscosities may need a different average torque generated from the torque spring 136 if the same dosing is desired. The average torque output may be controlled by adjusting the thickness of the band used for the torque spring 136 (e.g., the axial length of the torque spring 136 when disposed in the device) and maintaining the same number of working turns. Doing so may allow different springs to be used with the same configuration as the device and have similar injection times while the volume and/or viscosity of the drug may be modified.

In some examples, the energy (EFLOW) required to expel the medicament 113 through a needle 114 is determined by any combination of the drug volume, viscosity, needle flow path dimensions, and the targeted dosing time. The energy (ESPRING) that the torque spring 136 delivers may be determined by any combination of the number of working turns (N) and the average spring torque during the working turns (T). The energy delivered by the spring may be calculated using the following formula: ESPRING=2*ττ*N*T. If frictional losses are excluded in the system, the following relationship exists: EFLOW=ESPRING−2*ττ*N*T. Accordingly, the following relationship results: EFLOW/(2*ττ)=N*T. In other words, to have sufficient energy in the torque spring 136 to expel a given drug in a given volume through a given needle in a given time, the product (N*T) remains constant, and thus the higher torque may be converted to fewer working turns.

The threaded interface between the plunger rod 131 and the nut 122 provides a translation between the input torque of the torque spring 136 and the output axial force. By providing a torque spring 136 with a high turn count, it will have a lower overall torque as well as a smaller change in start and end torque as compared to a linear spring having comparable gearing specifications or other torsion springs with few turns and a lower pitch. Additionally, the threads of the plunger rod 131 and the nut 122 can have a lower pitch due to the increase in turn count, while still achieving the same linear motion of the plunger rod assembly 130. If the thread pitch is low, a smaller input torque is necessary to provide the same output force as a high pitch thread and high torque spring. Accordingly, the high turn count (e.g., between approximately 1 and approximately 30 turns), low torque system described herein allows for reduced activation forces, as the activation force is directly related to the input torque that must be used to drive the plunger rod assembly 130. Additionally, internal structural forces required to resist the torque from the torque spring 136 during storage (e.g., prior to use) is reduced, thus allowing for smaller injector designs to be used and for less expensive raw materials to be used. Additionally, the threaded interface between the plunger rod 131 and the nut 122 allows the threaded plunger rod 131 to be adjusted to accommodate for varying quantities of medicament stored in the syringe barrel 112. If necessary, the threaded plunger rod 131 may be initially installed at a lower position in injectors 100 having lesser drug product volumes disposed in the syringe barrel 112. Accordingly, the number of unique components is reduced, and variation management is simplified. The threaded plunger rod 131 may also be adjustably installed at various depths during the manufacturing and/or assembly process as needed.

The damper mechanism 140 is operably coupled to a portion of the drive assembly 120 (e.g., the plunger rod guide 126) and the housing 102. The damper mechanism 140 acts to dampen the effect of the torque spring 136 on the drive assembly 120.

Generally, to activate the device, a user presses the device 100 against their skin, thereby causing the trigger ring 124 to disengage from the nut 122 and/or the plunger rod guide 126. Such disengagement allows the plunger rod guide 126 to rotate relative to the trigger ring 124. Because the torque spring 136 is in a wound or compressed state, the torque spring 136 will begin to unwind, thereby causing the plunger rod guide 126 to rotate. This rotation in turn causes the plunger rod 131 to rotate, which, due to the threaded interface between the plunger rod 131 and the nut 122, causes the plunger rod 131 and the plunger 133 to advance towards the proximal end 102a of the housing 102, thereby inserting the needle or cannula 114 and administering the medicament 113. As a non-limiting example, U.S. Provisional Application No. 62/719,367, filed on Aug. 17, 2018, describes an activation process and components of the drive assembly in further detail and accordingly is incorporated by reference herein in its entirety.

In the illustrated example of FIGS. 1 and 2, the damper mechanism 140 includes the damper member 144, the frame member 142, and the damper fluid 146 disposed within the damper fluid chamber 148 between the frame member 142 and the damper member 144. The damper member 144 may be coupled to the plunger rod guide 126 via any number of approaches such as, for example, via a friction fit or threaded engagement. For example, as shown in FIG. 3, a portion of the plunger rod guide 126 may be received in the central bore 162B of the body 162 of the damper member 144.

As previously mentioned, relative rotation between components of the damper mechanism 140 causes the damper fluid 146 to dampen the torque applied by the drive assembly 120. Specifically, in this example, the damper member 140 rotates relative to the frame member 142 when the plunger rod guide 122 rotates. A torque from the torque spring 136 exists between the damper member 144 and the frame member 142, thereby causing the system to accelerate from rest thus increasing speed. During relative rotation, the damper fluid 146 experiences shear stress due to rotation of the damper member 144. In the disclosed example, the damper fluid 146 thus exerts an opposite acting reaction torque on the drive assembly 120 and, in particular, the plunger rod guide 126 of the drive assembly 120. The speed of the drive assembly 120 increases until the opposite acting damper torque has been built up to the same level as the dosing torque and equilibrium is reached. This equilibrium occurs at a specific speed and torque and is dependent on a number of factors such as, for example, geometry of the damper mechanism 140, fluid properties of the damper fluid 146, and the torque profile of the torque spring 136. Other examples are possible.

So configured, the damper mechanism 140 has a relatively simple design using minimal parts to reduce assembly and component costs and complexity. The damper mechanism 140 may be easily assembled, filled, and tested on a separate assembly line prior to being inserted into the device 100. In some examples, it may also be of interest to have a robust and stable damper mechanism 140. There are a number of parameters that may affect the performance of the damper mechanism 140, and by reducing the influence of these parameters may further increase the stability of the damper mechanism 140.

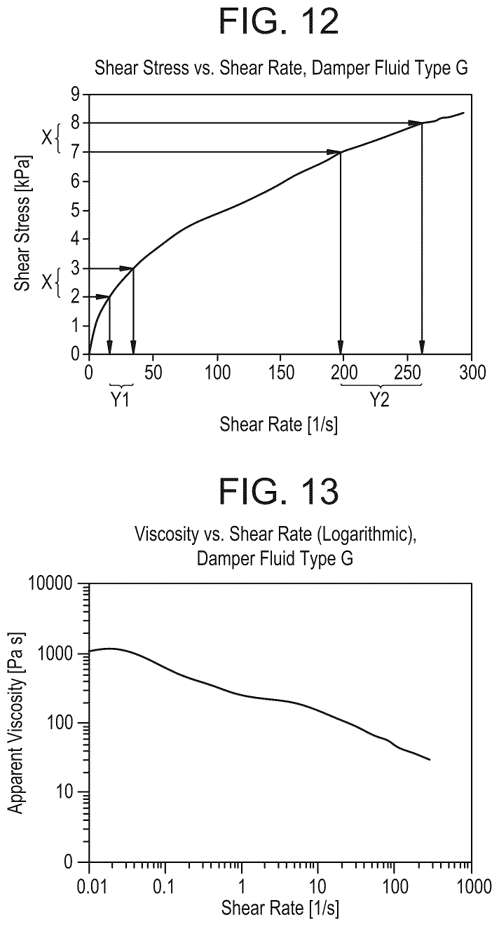

For example, and as previously noted, a damper fluid 146 having a low variation in viscosity as a function of temperature may be selected that have shear thinning properties. The shear stress in the damper fluid 146 is directly related to the damping torque. To obtain a relative constant and predictable speed at a certain needed damping torque, it is desired to have a change in input torque (and thereby shear stress) cause a minimal change in shear rate. In some examples, and as illustrated in FIG. 12 that depicts shear stress as a function of shear rate for a damper fluid type “G”, this may be best obtained by having a design with a shear rate in the lower end, as the variation in shear rate, y, at a given input torque interval is less in this area. It is noted that the provided curve in FIG. 12, and the values illustrated therein, only represent an example curve, and accordingly other curves may be used. FIG. 13 illustrates the apparent viscosity of the damper fluid type G. The shear thinning properties can be seen by the decrease in the apparent viscosity with the increase in shear rate.

Another parameter that may impact robustness and stability of the damper mechanism include a large gap at a small diameter. The shear rate level is designed to and influenced by the dimensions of the damper mechanism 140. The size of the gap that defines the damper fluid chamber 148 impacts the shear rate. The part tolerances can impact the size of the damper fluid chamber 148 the least amount if the nominal damper fluid chamber 148 size is as large as possible and if the damper fluid chamber 148 is placed at the smallest possible diameter.

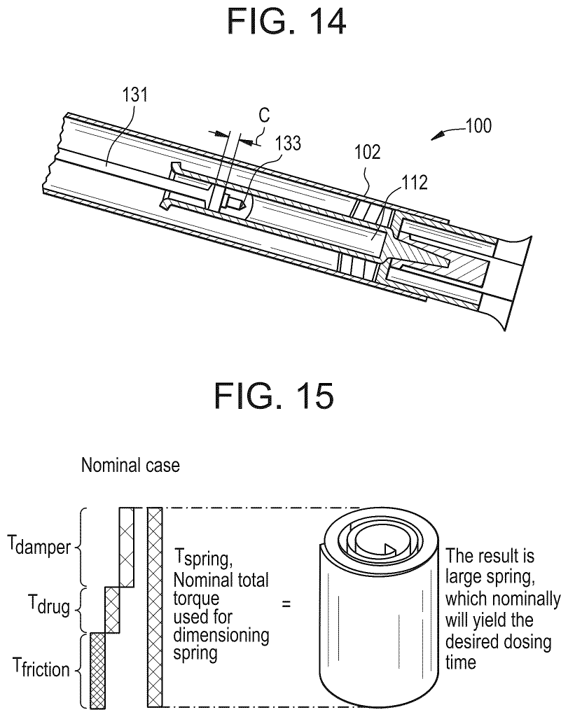

Further, with brief reference to FIG. 14, the described damper mechanism 140 may allow for significant clearances “C” (e.g., approximately 10 mm or more) between the plunger rod 131 and the plunger 133 without risking breakage of the syringe barrel 112 or other components of the device 100 upon its activation and upon impact between the plunger rod 131 and the plunger 133. These devices may be adapted to extrude at least approximately 1 ml of medicament 113 having a viscosity of at least approximately 4 cP. Such large clearances advantageously reduce platform complexity, inventory variations, and/or process controls. The damper mechanism 140 also provides for a better user experience when compared to devices without a damper mechanism, where the impact shock, feel, and sound may startle a user.

Turning to FIGS. 15-17, in some examples, it may be advantageous to construct the syringe barrel 112 out of different materials. Because of high friction variation in containers constructed from some materials, there may be significant variance in delivery times. The friction between the plunger and the syringe barrel may result in substantial variation, especially when the syringe is constructed from a polymeric material. As noted, the force required for expelling the drug through the needle in a specified time, which is directly linked to the axial plunger velocity, varies with the viscosity of the drug. For high-viscosity drugs (e.g., above 10-15 cP), the force requirement is high, and for low viscosity drugs, the force requirement is low. The force is also dependent on the velocity at which the drug is expelled. During dosing, an equilibrium dosing velocity is achieved where the velocity-dependent resistance in the system matches the input torque from the power source. However, the range in which the frictional forces vary in the system is constant regardless of the viscosity of the drug. Consequently, the ratio between frictional forces and drug expulsion force becomes high in the case of low-viscosity drugs. Further, since a high variability in the frictional forces is expected for polymer syringe barrels, the remaining torque from the spring for expelling the drug can either be too high or too low, resulting in too fast or too slow dosing time. This can result in either unacceptably high variations in dosing times or that the device stalls altogether.

The use of a damper mechanism addresses these inconsistencies by acting as a buffer of excess torque. The velocity of the dosing mechanism is the result of a mechanical equilibrium, in which the friction in the system, the torque required to expel the drug, and the torque acting on the mechanical damper is equal to the total input torque from the power source. Because the non-constant torques, the damper torque and the torque required the expel the drug added, become more dominant than the frictional forces, the variation in the frictional forces will have less relative impact on the available torque for the expulsion, and will therefore affect the velocity modestly. Generally, whenever the resistance in the device increases—be it during dosing due to friction and component tolerances, or because of a higher drug viscosity—the velocity in the device decreases. Because of the velocity-dependence of the damper, however, an infinitesimal decrease in velocity leads to a lower damping torque, which in turn frees up available torque for overcoming the increased resistance.

As shown in FIGS. 15-17, the variances between plunger friction, torque required to expel the drug, and torque absorbed by the damper are added to provide a nominal input torque requirement. It is noted that the torque contributions in the device are not limited to these provided terms. Because the damper dissipates a substantial amount of torque, the spring is dimensioned larger than if no damper was used. The velocity-dependent terms (i.e., T_{damper} and T_{drug}) dissipate the majority of the energy in the device.

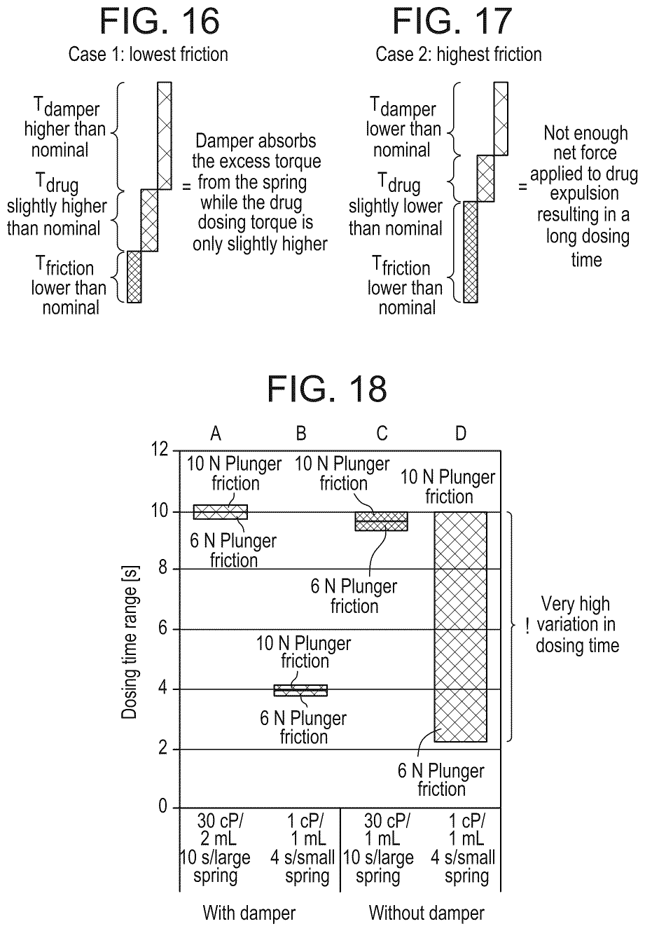

Because a small decrease in velocity corresponds to a large decrease in damper torque (and vice-versa), only a minor change in available torque for drug expulsion is observed. This is illustrated in FIGS. 16 and 17: in case 1, shown in FIG. 16, the friction is in the lower end of the expected range that results in the viscous terms to increase in magnitude because of more available torque, where the damper term will absorb the most torque while the torque available for drug expulsion increases only slightly. This results in only a slightly faster dosing time. Conversely, in case 2 illustrated in FIG. 17, an increase friction results in the damper torque decreasing substantially, while the available torque for drug expulsion decreases only slightly, thus yielding only a slightly longer dosing time. In devices without damper mechanisms, a substantial percentage of the input torque is used for overcoming the friction in the system. In variation in friction will directly add or subtract substantially on the available torque for the expulsion part. High fluctuations can therefore be expected at dosing time.

Turning to FIG. 18, an example of the high sensitivity is illustrated by model calculations. The first two columns A and B illustrate the range of dosing times for a range of friction values where a mechanical damper mechanism is used. Both the high viscosity (column A) and low viscosity (column B) drug variant devices exhibit a narrow variation in dosing time thanks to the damper. For the variants without a damper (columns C and D), the variability is similarly low for high viscosity drug variants. This is attributed to the high proportion of the input torque spent on expelling the drug relative to the torque used for overcoming the constant friction. However, for the low viscosity drug variant (column D), where no damper is used, the dosing time varies dramatically with the friction variance. In addition to the high dosing time sensitivity towards friction variability, in terms of a device platform, any change in drug viscosity will substantially change the input torque requirements if no damper is used. Therefore, in order to achieve the desired window of doing times, a higher number of power springs would be required. Having a damper, on the other, introduces the buffering phenomenon at the expense of a slightly larger spring.

Additionally, certain materials may impact these forces. For example, when using a glass syringe, due to the siliconization of the barrel and the stopper, there may be a lower glide force, and lower variation of the glide force relative to plastic syringes. When administering drugs having high viscosities, the resistance of flow through the needle tends to be the largest contributor to overall injection times. However, when administering drugs having low viscosities and volumes, the glide force (and its relative variability) can be a large contributor to the total required force in the system.

So configured, the above damper designs can reduce the number of required spring variants in an autoinjector platform, can improve consistency of dose times for users, and can reduce risks of syringe breakages. Because minor variations in spring performance and/or drug viscosity can have a significant impact when using low-viscosity drugs, the damper mechanisms described herein slows all dose times, thereby requiring fewer spring variants. When using drugs having high viscosities, the damper mechanisms described herein have a greater effect on the impact speed of the plunger rod, especially when administering low volume drug products. The damper mechanism will reduce the impact speed of the plunger rod to a safer level to reduce the risk of damaging the syringe. The damper mechanisms described herein require fewer parts, thereby assisting in assembly and cost reduction. Additionally, the damper mechanisms described do not rely on surface friction and relatively complex moving mechanisms and thus further reduce system complexities.

The present disclosure relates to a damper mechanism 140 for a drug delivery device 100 that has a damper fluid chamber 148 with an overfill section 152 that receives excess damper fluid 146 so that the level of damper fluid 146 in the damper fluid chamber 148 does not change or only minimally effects the resulting damper torque. This may allow the damper mechanism 140 to perform more consistently, while at the same time lowering tolerance requirements on both the injection molded parts, the damper fluid, the filling process, and the assembly process.

For combination product auto injectors, where a primary container is combined with a mechanical device, clearances may exist in the drive mechanism and injection system, e.g., between the plunger and washer. As the spring driven mechanism may not meet resistance in this clearance travel, the mechanism may accelerate fast and then shortly after, decelerate fast, when no more clearance exists in the system.

A damper mechanism may be implemented in the auto injector to control the movement of the drive assembly. Speed dependent damper mechanisms may be desirable as these devices react and create a force balance, thereby creating a controlled movement during the clearance travel at a designed acceptable speed level. At dosing speed, which is at relatively much lower speed, the same damper mechanism may create minimal resistance to the system, which may allow the power source to the system to be as small as possible. Subsequently, the auto injector mechanism may be as small as possible.

Speed dependent damper mechanisms may be purely mechanical, but such devices may take up large amounts of space in the device to have a sufficient damper effect. This may make the device too large.

The fluid damper may allow the damper mechanism to be considerably smaller in size than comparable known purely mechanical dampers. However, resistance at low speeds may still increase, which leads to added costs related to a larger spring and more durable parts to cope with the forces. Additionally, variation in fill level of the damper fluid during the filling and assembly process may affect the damper mechanism.

In some embodiments, the damper mechanism 140 has a damper fluid chamber 148 that includes an overfill section 152. The overfill section 152 contains excess damper fluid. The overfill section 152 is sized so that excess damper fluid may have little to no effect on the rotational movement.

The overfill section 152 of the damper fluid chamber 148 is configured to reduce variance in the resistance that the damper fluid exerts to rotational input and to reduce/eliminate the risk of damper fluid 146 moving out of the damper fluid chamber 148. The risks of damper fluid 146 being present outside the intended areas may include altering friction in adjacent mechanical interfaces or eventually escaping to outer surfaces where the damper fluid 146 may alter the visual appearance of the products.

Both of the above objectives maybe challenged by several factors that affect the effective fill level in the chamber 148 of a viscous damper fluid 146. For example, part tolerances of the enclosing chamber 148 (diameters, heights, surface topology, draft angle, roundings) may affect the effective fill level in the chamber 148. Damper fluid fill volume (process tolerances, damper fluid density, temperature) may affect the effective fill level in the chamber 148. Air bubbles trapped in the damper fluid (both during dispensing and after dispensing and during assembly of the rotation and stationary parts) may affect the effective fill level in the chamber 148. Uneven damper fluid distribution (off-center dispensing of fluid, non-aligned stationary and rotating part during damper module assembly, topology of parts surfaces in contact with damper fluid) may affect the effective fill level in the chamber 148.

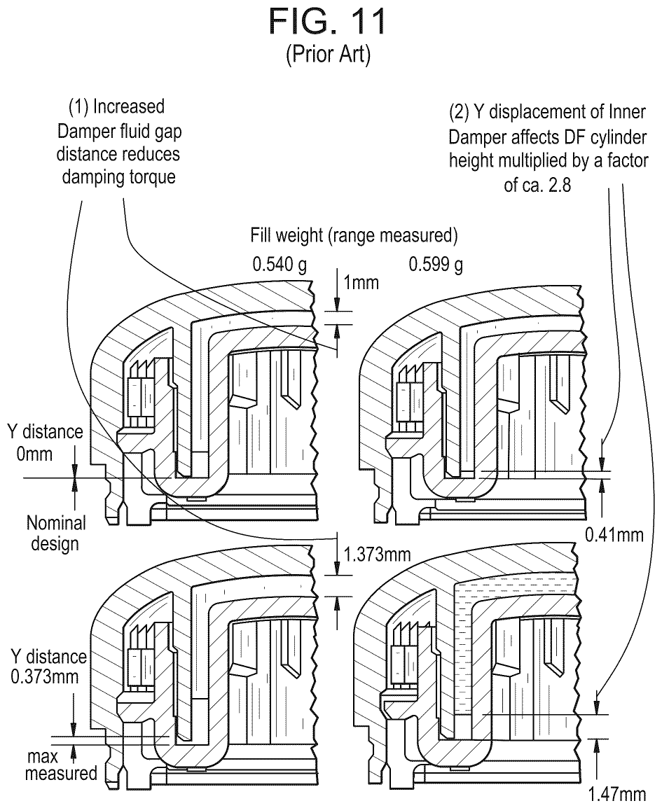

In FIG. 11, variations in fill level caused by assembly variation or varying a relative position between a frame member and a damper member of a drug delivery device are shown. For example, as shown in FIG. 11, an increased gap in a damper fluid chamber can reduce the damping torque applied to the drive assembly. In another example, as shown in FIG. 11, vertically displacing the damper mechanism from the frame member can affect the torque applied to the drive assembly. Accordingly, FIG. 11 illustrates how the relative positioning of the frame member and damper member is one of several causes that can vary the effective fill level of damper fluid for a damper mechanism, such as the damper mechanism of FIGS. 9A-9C and the damper mechanism of FIGS. 10A-10C. The damper mechanism 140 of FIGS. 8A-8C mostly or fully mitigates the variations in fill level of damper fluid caused by assembly variation by providing an overfill section 152 in the damper fluid chamber 148. The overfill section 152 accommodates excess damper fluid from the main section 150 and, thereby, minimizes the effect of variation in fill level of damper fluid on the amount of opposing force or torque applied to the drive assembly. The damper mechanism in FIG. 11 does not include an overfill section that may cause gaps or separation between components to vary.

As discussed above, FIGS. 8A-8C, 9A-9C, and 10A-10C show how different fill levels of damper fluid caused by various variation drivers may manifest in the different embodiments: a damper mechanism with a spillover region, FIGS. 9A-9C), a damper mechanism without any overfill chamber, FIGS. 10A-10C), and finally the damper mechanism 140 of FIGS. 1-5 and 8A-8C). In some embodiments, any excess damper fluid may be mostly or completely passivated in that the damper fluid may not increase/may minimally increase the resulting damper torque. An example of this may be an overfill section 152 of the damper fluid chamber 148 sized so that excess damper fluid extrudes into a void in the overfill section 152 where there is no surface contact of the excess damper fluid to either of the sides of the overfill section 152. This may be possible with extremely high viscous damper fluid.

In some embodiments, the overfill section 152 increases the thickness of the damper fluid chamber 148 relative to the main section 150 to effectively reduce the shear rate and thus the resulting specific torque contribution. In some embodiments, the thickness 152T of the overfill section 152 is increased by increasing the diameter of the frame member 142. In other embodiments, the thickness 152T of the overfill section 152 is increased by decreasing the diameter of the damper member 144.

In other embodiments, the effects may be achieved by reducing/eliminating the ability of the excess damper fluid to exert torque on the rigid parts, i.e., the frame member 142. This may be achieved by facilitating intrusion of air into the overfill section 152, which may abruptly degrade the resulting torque in the main section 150 and effectively remove the surface contact). In other embodiments, the surface area in the overfill section 152 may be altered to reduce adhesion to the fluid promoting slip stick. In other embodiments, the surface area in the overfill section 152 may be coated with a low vicious intermediate fluid to serve as a lubricating boundary layer reducing the resulting torque of the excess damper fluid. In other embodiments, one or both the rigid parts may be lined with one or more rigid rotating part to effectively separating the excess damper fluid from the rotation.

For example, as shown in the damper mechanism of FIGS. 9A-9C where the damper mechanism has a spillover region, if excess damper fluid flows into out of the fluid damper chamber into the spillover region, the damper torque may increase significantly, for example, as shown in FIG. 7. The spillover region may cause an excessive damper torque increase when the spillover region is utilized as this region is farther away from the center axis L, thereby increasing the moment arm length as well as shear rate in the fluid in this region compared to fluid in the damper fluid chamber. The utilization of the spillover region therefore further exacerbates the resulting torque increase from relative low volumes of excess fluid. In FIG. 7, the graph illustrates the possible effect of the “spillover” region of FIGS. 9A-9C. The near vertical section right after the nominal fill level may be caused by fluid spilling out of the chamber before reaching the spillover region. Moreover, as shown in the damper mechanism of FIGS. 9A-9C, there is nearly no axial gap between a terminal end of the frame member and a band member of the damper member, which may significantly increase the shear rates and, thus, further increase the damper torque applied to the drive assembly.

In some embodiments, the variation in fill level can be accounted for during production and assembly of the damper mechanism. For example, during production and/or assembly of the damper mechanism, the nominal performance of each component of the damper mechanism can be measured and adjusted such that the resulting torque exerted by the damper mechanism is consistent for all components of the damper mechanism. Monitoring the performance of the damper mechanism during production and assembly may allow for an accurate, precise, and stable performance (e.g., resulting torque) of the damper mechanism even if, for example, excess damper fluid flows out of the overfill section 152 into the gap 168 and/or the space 169. Additionally, monitoring the performance of the damper mechanism during production and assembly may allow the damper mechanism to exert precise and accurate amount of torque at any given fill level in the damper fluid chamber and, also, compensate for bias in other variance factors such as variance in the geometry of individual components of the damper mechanism, variance in the viscosity of the damper fluid, or the like.

The above description describes various assemblies, devices, and methods for use with a drug delivery device having a damper mechanism. It should be clear that the assemblies, drug delivery devices, damper mechanisms, or methods can further comprise use of a medicament listed below with the caveat that the following list should neither be considered to be all inclusive nor limiting. The medicament will be contained in a reservoir. In some instances, the reservoir is a primary container that is either filled or pre-filled for treatment with the medicament. The primary container can be a cartridge or a pre-filled syringe. The primary container may be a needle assembly comprising a syringe barrel.

For example, the drug delivery device or more specifically the reservoir of the device may be filled with or the device can be used with colony stimulating factors, such as granulocyte colony-stimulating factor (G-CSF). Such G-CSF agents include, but are not limited to, Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In various other embodiments, the drug delivery device may contain or be used with various pharmaceutical products, such as an erythropoiesis stimulating agent (ESA), which may be in a liquid or a lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (cpoctin alfa, or erythropoietin); GLP-1, Avonex® (interferon beta-1a); Bexxar® (tositumomab, anti-CD22 monoclonal antibody); Betascron® (interferon-beta); Campath® (alemtuzumab, anti-CD52 monoclonal antibody); Dynepo® (cpoctin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoctin alfa); Erbitux® (cctuximab, anti-EGFR/HER1/c-ErbB-1); Genotropin® (somatropin, Human Growth Hormone); Herceptin® (trastuzumab, anti-HER2/neu (crbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (cpratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoctin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, cdrecolomab); Trabio® (lerdelimumab); TheraCim hR3 (nimotuzumab); Omnitarg (pertuzumab, 2C4); Osidem® (IDM-1); OvaRcx® (B43.13); Nuvion® (visilizumab); cantuzumab mertansine (huC242-DM1); NeoRecormon® (cpoctin beta); Neumega® (oprelvekin, human interleukin-11); Orthoclone OKT3® (muromonab-CD3, anti-CD3 monoclonal antibody); Procrit® (cpoctin alfa); Remicade® (infliximab, anti-TNFα monoclonal antibody); Reopro® (abciximab, anti-GP IIb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-cotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGFIR mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PDImAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooc), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl|acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (cculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omccamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with rocatinlimab (AMG 451), a human anti-OX40 monoclonal antibody that is expressed on activated T cells and blocks OX40 to inhibit and/or reduce the number of OX40 pathogenic T cells that are responsible for driving system and local atopic dermatitis inflammatory responses. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein (a), also known as Lp (a), or another product containing a small interfering RNA (siRNA) that lowers lipoprotein (a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITER). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BITER (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BITE® (bispecific T cell engager) molecule. In some embodiments, the drug delivery device may contain or be used with AMG 673 or another product containing a half-life extended (HLE) anti-CD33×anti-CD3 BITER (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, methods, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention. For example, components described herein with reference to certain kinds of drug delivery devices, such as autoinjector drug delivery devices or other kinds of drug delivery devices, can also be utilized in other kinds of drug delivery devices, such as on-body injector drug delivery devices.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A damper mechanism for a drug delivery device, the damper mechanism comprising:
a frame member;
a damper member operably coupled to a drive assembly of the drug delivery device;
a damper fluid chamber formed between at least a portion of the frame member and the damper member; and
a damper fluid disposed within the damper fluid chamber, wherein upon activating the drive assembly of the drug delivery device, the frame member and the damper member rotate relative to each other and the damper fluid exerts an opposing force on at least one of the frame member and the damper member, and
wherein the damper fluid chamber includes a main section and an overfill section, the overfill section being configured to receive excess damper fluid from the main section to reduce an effect a variation in fill level in the damper fluid chamber has on the opposing force exerted on at least one of the frame member and the damper member, the main section having a first thickness defined between an inner surface of the frame member and an outer surface of the damper member, and the overfill section having a second thickness defined between the inner surface of the frame member and the outer surface of the damper member that is greater than the first thickness of the main section.

2. The damper mechanism of claim 1, wherein the frame member extends circumferentially around the longitudinal axis to define a cavity, the frame member comprising an end wall and a cylindrical protrusion that extends axially away from the end wall relative to a longitudinal axis to a terminal end spaced apart axially from the end wall.

3. The damper mechanism of claim 2, wherein the damper member is located in the cavity formed by the frame member to form the damper fluid chamber between at least the portion of the frame member and the damper member.

4. The damper mechanism of claim 2, wherein the cylindrical protrusion of the frame member comprises:
a base section that extends axially away from the end wall, and
a tip section that extends axially away from the base section to the terminal end,
wherein the base section of the cylindrical protrusion of the frame member has a first thickness and the tip section of the cylindrical protrusion of the frame member has a second thickness that is less than the first thickness of the base section.

5. The damper mechanism of claim 1, wherein the damper member comprises a body that extends circumferentially around the longitudinal axis to define the outer surface of the damper member, and wherein a thickness of the body proximate the main section is greater than a thickness of the body proximate the overfill section.

6. The damper mechanism of claim 1, wherein the damper member comprises:
a body that extends circumferentially around the longitudinal axis to define the outer surface of the damper member;
an outer ring that extends circumferentially around the body and spaced apart radially from the body relative to the longitudinal axis to define a channel therebetween; and
a band member that extends radially between and interconnects the body and the outer ring to form a bottom of the channel,
wherein the cylindrical protrusion of the frame member extends axially from the housing into the channel between the outer surface of the body and an inner surface of the outer ring, and
wherein the damper fluid chamber is defined between the outer surface of the body and the inner surface of the frame member.

7. The damper mechanism of claim 6, wherein the terminal end of the frame member is spaced apart axially from the band member of the damper member to define a gap therebetween.

8. The damper mechanism of claim 7, wherein the gap is free of any excess damper fluid, or a space defined directly between the frame member and the outer ring of the damper member is free of excess damper fluid.

9. The damper mechanism of claim 6, wherein the body of the damper member comprises:
an end wall spaced apart axially from the frame member to define a portion of the damper fluid chamber; and
an annular wall that extends axially away from the end wall and extends circumferentially around the longitudinal axis to define a bore, the bore being configured to receive a portion of the drive assembly of the drug delivery device.

10. The damper mechanism of claim 1, wherein the damper member comprises:
a body that extends circumferentially around the longitudinal axis to define the outer surface of the damper member;
an outer ring that extends circumferentially around the body and spaced apart radially from the body relative to the longitudinal axis to define a channel therebetween; and
a band member that extends radially between and interconnects the body and the outer ring to form a bottom of the channel,
wherein the cylindrical protrusion of the frame member extends axially from the housing into the channel between the outer surface of the body and an inner surface of the outer ring,
wherein the damper fluid chamber is defined between the outer surface of the body and the inner surface of the frame member.

11. The damper mechanism of claim 10, wherein the body of the damper member comprises at least one of:
an end wall spaced apart axially from the frame member to define a portion of the damper fluid chamber;
an annular wall that extends axially away from the end wall to the band member and extends circumferentially around the longitudinal axis to define a bore; or
an extension that extends axially from the end wall of the body opposite the annular wall and engages the frame member.

12. The damper mechanism of claim 1, wherein the housing comprises:
a housing shell that extends along the longitudinal axis; and
a housing end cap coupled to the housing shell opposite the proximal end,
wherein the frame member is formed integrally with the housing end cap of the housing.

13. The damper mechanism of claim 1, wherein the overfill section of the damper fluid chamber is coated with a low vicious intermediate fluid to serve as a lubricating boundary layer reducing the resulting torque of the excess damper fluid.

14. A drug delivery device comprising:
a housing having a proximal end, a distal end, and a longitudinal axis extending between the proximal end and the distal end thereof;
a needle assembly at least partially disposed within the housing at the proximal end thereof, the needle assembly comprising a syringe barrel containing a medicament and a needle or a cannula;
a drive assembly at least partially disposed within the housing and operably coupled to the needle assembly to urge the medicament through the needle or cannula; and
a damper mechanism at least partially disposed within the housing adjacent to the distal end thereof, the damper mechanism being operably coupled to the drive assembly and the housing, wherein upon activating the drive assembly, the damper mechanism dampens an effect thereof, the damper mechanism comprising:
a frame member;
a damper member operably coupled to the drive assembly;
a damper fluid chamber formed between at least a portion of the frame member and the damper member; and
a damper fluid disposed within the damper fluid chamber, wherein upon activating the drive assembly of the drug delivery device, the frame member and the damper member rotate relative to each other and the damper fluid exerts an opposing force on at least one of the frame member and the damper member, and
wherein the damper fluid chamber includes a main section and an overfill section, the overfill section being configured to receive excess damper fluid from the main section to reduce an effect a variation in fill level in the damper fluid chamber has on the opposing force exerted on at least one of the frame member and the damper member, the main section having a first thickness defined between an inner surface of the frame member and an outer surface of the damper member, and the overfill section having a second thickness defined between the inner surface of the frame member and the outer surface of the damper member that is greater than the first thickness of the main section.

15. The drug delivery device of claim 14, wherein the frame member comprises:
an end wall that defines a portion of the distal end of the housing; and
a cylindrical protrusion that extends axially away from the end wall toward the proximal end relative to the longitudinal axis to a terminal end spaced apart axially from the end wall,
wherein the cylindrical protrusion of the frame member varies in thickness as the cylindrical protrusion extends axially away from the end wall to the terminal end.

16. The drug delivery device of claim 15, wherein the cylindrical protrusion of the frame member comprises:
a base section that extends axially away from the end wall; and
a tip section that extends axially away from the base section to the terminal end,
wherein the base section of the cylindrical protrusion of the frame member has a first thickness and the tip section of the cylindrical protrusion of the frame member has a second thickness that is less than the first thickness of the base section.

17. The drug delivery device of claim 14, wherein the damper member comprises:
a body that extends circumferentially around the longitudinal axis to define the outer surface of the damper member;
an outer ring that extends circumferentially around the body and spaced apart radially from the body relative to the longitudinal axis to define a channel therebetween; and
a band member that extends radially between and interconnects the body and the outer ring to form a bottom of the channel,
wherein the frame member extends axially from the housing into the channel between the outer surface of the body and an inner surface of the outer ring, and
wherein the damper fluid chamber is defined between the outer surface of the body and the inner surface of the frame member.

18. The drug delivery device of claim 17, wherein the terminal end of the frame member is spaced apart axially from the band member of the damper member to define a gap therebetween.

19. The drug delivery device of claim 18, wherein the gap is free of any excess damper fluid, and wherein there is no excess damper fluid in a space defined between the frame member and the outer ring of the damper member.

20. The drug delivery device of claim 14, wherein the housing comprises:
a housing shell that extends along the longitudinal axis, and
a housing end cap coupled to the housing shell opposite the proximal end,
wherein the frame member is formed integrally with the housing end cap of the housing.
